(19) United States
(12) Patent Application Publication
(10) Pub. No.: US 2007/0185383 A1
(43) Pub. Date: Aug. 9, 2007

(54) Tapered endoscopic protective sheath

(75) Inventors: Tim Mulhern, Natick, MA (US); Stephen Martone, Nashua, NH (US)

Correspondence Address: WOLF, BLOCK, SCHORR & SOLIS-COHEN LLP, 250 PARK AVENUE, NEW YORK, NY, 10177, US

(73) Assignee: Vision-Sciences, Inc., Natick, MA (US)

(21) Appl. No.: 11/349,636

(22) Filed: Feb. 8, 2006

(51) Int. Cl. A61B 1/00

(52) U.S. Cl. 600/121; 600/101; 600/153; 600/124

(57) ABSTRACT

A sheath apparatus for an invasive probe. The sheath apparatus includes a protective sheath, adapted to receive an insertion tube of an endoscope. The protective sheath is tapered over most of its length with a tapering of less than 0.2 degrees, but more than 0.001 degrees.

### FIELD OF THE INVENTION

The present invention relates to sheaths for medical apparatus.

### BACKGROUND OF THE INVENTION

Endoscopes are used to view internal tissue of humans, and for many other tasks. As sterilization of endoscopes is relatively difficult, disposable sheaths which cover an endoscope are used to isolate the endoscope from the patient tissue, so as to avoid time-consuming cleaning and disinfection processes. In some cases it is desired to have one or more channels run along the endoscope. These channels may be used, for example, to pass tools and fluids to the tip of the endoscope.

U.S. Pat. No. 5,667,068 to Weaver, the disclosure of which is incorporated herein by reference, describes a protective cover for an endoscope used in the storage of the endoscope.

U.S. Pat. No. 4,062,239 to Fowler et al., the disclosure of which is incorporated herein by reference, describes a protective cover for medical probes, such as thermometers, with an inner sheath which is tapered on a sensing end of the inner sheath.

For relatively long endoscopes of more than 25 or even 50 centimeters, mounting the sheath on the endoscope may be problematic, since for sterility purposes it is desired to perform the mounting without touching surfaces of the sheath that come in contact with the patient's body fluids and/or tissue. In some cases, the sheath has a proximal rigid port, which is held by a physician mounting the sheath on the insertion tube.

U.S. Pat. No. 5,520,607 to Frassica et al., the disclosure of which is incorporated herein by reference, describes a holding tray assembly for holding an endoscopic sheath during installation of the sheath onto an insertion tube of the endoscope.

Still, in some cases, if the sheath is not pulled fast enough over the insertion tube, the sheath may bend over and require a corrective handling of a physician in order to continue the insertion of the endoscope into the sheath, without a risk of tearing. This problem becomes more severe when the sheath is part of a sheath assembly including working tubes and/or a plurality of sheaths, as there are more cases in which a portion of the sheath assembly moves out of place during a loading of the endoscope into the sheath.

U.S. Pat. No. 6,224,543 to Gammons et al., the disclosure of which is incorporated herein by reference, describes a sheath arrangement for mounting of a sheath on a probe. The sheath is mounted in a mostly inverted orientation on a flexible carrier. In order to mount the sheath on the probe, the probe is pushed into a non inverted tip of the sheath, so as to disengage the sheath from the carrier and un-invert the sheath. In order to facilitate the straightening (i.e., un-inversion), the sheath is tapered.

U.S. Pat. No. 6,755,789 to Stringer et al., the disclosure of which is incorporated herein by reference, describes a protective sheath, for an elongated medical probe. The protective sheath is packaged with half the sheath folded back everted inside-out over the remaining part of the sheath. The everted sheath is rolled into a small pouch. The sheath may be tapered, to simplify the opening of the sheath.

### SUMMARY OF THE INVENTION

An aspect of some embodiments of the present invention relates to a tapered protective sheath for a medical probe which includes a proximal port more rigid than the protective sheath. The relatively rigid port of the protective sheath optionally prevents the proximal end of the sheath, which is wide due to the tapering, from interfering with the operation of the medical probe. On the other hand, the tapering may allow a more easy insertion of the medical probe into the sheath. Although the sheath may be slightly baggy on the proximal portion of the medical probe, the advantages of the easy insertion may be considered to outweigh the disadvantages of being baggy.

An aspect of some embodiments of the present invention relates to a protective sheath of an elongated invasive medical probe, such as an endoscope or cystoscope, which is tapered along most (e.g., at least 60%, 80% or even 90%) of its length by a small amount of less than 1 degree or even less than 0.5 degrees. In some embodiments of the invention, the sheath has a taper degree of not more than 0.1 degrees or even less than 0.07 degrees. The invasive probe is optionally not tapered or is tapered to a smaller degree than the protective sheath.

The tapering is optionally of a substantial extent of at least 0.005 or even at least 0.01 degrees.

In some embodiments of the invention, the portions of the invasive probe inserted into the patient's body are covered by the sheath with a maximal difference between the inner diameter of the protective sheath and the outer diameter of the corresponding length of the probe smaller than 5 millimeters, or even smaller than 2 millimeters.

In some embodiments of the invention, the sheath is produced using an accurate production method with an accuracy resolution of less than 0.05 millimeters or even less than 0.01 millimeters, such as the heat forming method of U.S. Pat. No. 6,733,440 to Ailinger et al., the disclosure of which is incorporated herein by reference.

An aspect of some embodiments of the present invention relates to a sheath package of an elongated invasive medical probe, including a tapered protective sheath laid out in a non-everted orientation. While packaging in a non-everted orientation may result in larger packages and possibly considered more cumbersome, avoiding the eversion is considered worth the inconvenience.

An aspect of some embodiments of the present invention relates to a method of inserting an invasive probe into a tapered protective sheath, including providing the sheath in a laid out orientation and beginning insertion of the probe into the sheath when the sheath is laid out. The insertion of the tube may include moving the insertion tube relative to the sheath or moving the sheath relative to the tube.

In some embodiments of the invention, the protective sheath is longer than 25 centimeters or even longer than 50 centimeters.

There is therefore provided in accordance with an exemplary embodiment of the invention, a sheath apparatus for an invasive probe, comprising a protective sheath, adapted to receive an insertion tube of an endoscope, the protective sheath is tapered over most of its length with a tapering of less than 0.2 degrees, but more than 0.001 degrees.

Optionally, the protective sheath is tapered over most of its length with a tapering not greater than 0.1 degrees. Optionally, the protective sheath is tapered over most of its length with a tapering not greater than 0.06 degrees. Optionally, the protective sheath is thinner than 0.2 mm. Optionally, the sheath includes an additional external sheath, which surrounds the protective sheath. Optionally, the external sheath is tapered over most of its length with a tapering extent substantially the same as the protective sheath.

Optionally, the external sheath is tapered over most of its length with a tapering extent smaller than that of the protective sheath. Optionally, the sheath includes at least one channel tube between the protective sheath and the external sheath. Optionally, the protective sheath has a length of at least 300 millimeters

There is further provided in accordance with an exemplary embodiment of the invention, a method of inserting an endoscope into a protective sheath, comprising providing an insertion tube of an endoscope, covering the insertion tube with a protective sheath, which is tapered over most of its length, without everting the sheath in the process of covering the insertion tube.

Optionally, covering the insertion tube comprises covering with at least two sheaths, one of which is within the other. Optionally, all of the at least two sheaths are tapered at a same extent. Optionally, covering the insertion tube with a protective sheath, comprises inserting the endoscope into the protective sheath. Optionally, covering the insertion tube with a protective sheath, comprises pulling the protective sheath over the insertion tube.

There is further provided in accordance with an exemplary embodiment of the invention, a sheath apparatus for an invasive probe, comprising a tapered protective sheath, adapted to receive an insertion tube of an endoscope and a proximal port, which is rigid relative to the sheath, attached to a proximal end of the protective sheath.

Optionally, the sheath has a length of at least 250 millimeters.

There is further provided in accordance with an exemplary embodiment of the invention, a sheath package, comprising a sterile package; and a tapered protective sheath, adapted to receive an insertion tube of an endoscope, which is laid out over substantially its entire length without eversion, within the sterile package. Optionally, the package includes a proximal port, which is rigid relative to the sheath, attached to a proximal end of the protective sheath.

### BRIEF DESCRIPTION OF THE DRAWINGS

Exemplary non-limiting embodiments of the invention will be described with reference to the following description of the embodiments, in conjunction with the figures. Identical structures, elements or parts which appear in more than one figure are preferably labeled with a same or similar number in all the figures in which they appear, and in which:

FIG. 1A is a schematic view of an endoscope, in accordance with an exemplary embodiment of the invention;

FIG. 1B is a schematic sectional view of an endoscope with a sheath, in accordance with an exemplary embodiment of the invention;

FIG. 2A is a schematic side view of a sheath assembly, in accordance with an exemplary embodiment of the present invention; and

FIG. 2B is a cross-sectional view of the sheath assembly of FIG. 2A, in accordance with an exemplary embodiment of the present invention.

### DETAILED DESCRIPTION OF EXEMPLARY EMBODIMENTS

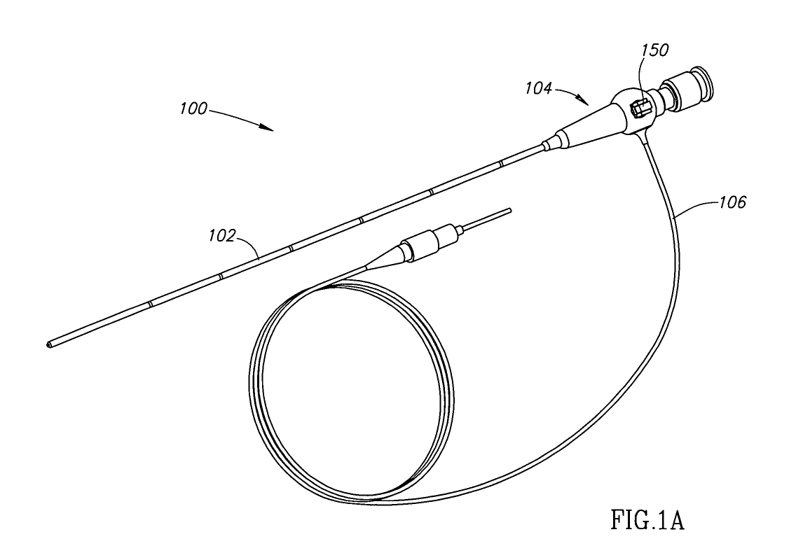

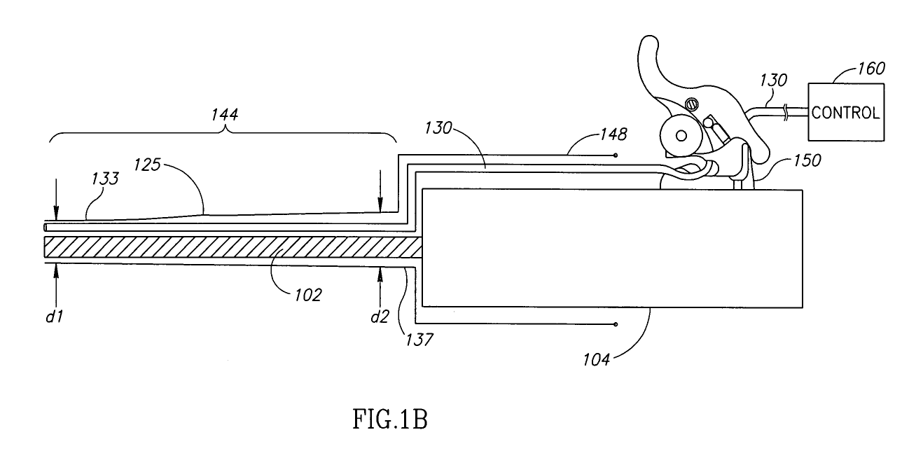

FIG. 1A is a schematic view of an endoscope 100, in accordance with an exemplary embodiment of the invention. Endoscope 100 includes an insertion tube 102, for insertion into a patient and a handle 104 for control of the insertion tube and/or for holding the endoscope by a physician. Insertion tube 102 may be adapted for insertion into any body cavity with which endoscopes are used, including, for example, the esophagus, colon and/or urethra. In an exemplary embodiment of the invention, endoscope 100 includes a cystoscope. An umbilical 106 optionally connects handle 104 to a light source (not shown). Insertion tube 102 optionally has a same diameter over its entire length.

In some embodiments of the invention, a valve 150 mounted on handle 104 is used to control the flow through a channel of a sheath covering endoscope 100.

FIG. 1B is a schematic sectional view of endoscope 100 with a sheath 125 including a channel tube 130 therein, in accordance with an exemplary embodiment of the invention. Sheath 125 optionally covers insertion tube 102 and at least a portion of handle 104. Channel tube 130 optionally extends proximally beyond sheath 125, for example for connection to a control station 160, which serves, for example, as a source of fluid (e.g., liquid and/or air) under positive or negative pressure. Channel tube 130 is optionally passed through valve 150 along its path between control station 160 and the distal end of insertion tube 102, so that a physician holding the endoscope by handle 104 can easily control the flow through tube 130, without aid of another person. In some embodiments of the invention, two, three or even more working channels are used with endoscope 100 according to the specific needs of the medical procedure being carried out.

Sheath 125 optionally comprises a thin film, which is bendable and flexible, so that the sheath does not substantially add to the cross-section area of the insertion tube and/or does not interfere with the manipulation of the insertion tube. A relatively rigid port 148 is optionally connected to sheath 125 at a proximal end of the sheath. Rigid port 148 may be held by a physician in mounting the sheath on insertion tube 102. Alternatively or additionally to holding port 148 by hand, a holding tray assembly, such as described in above mentioned U.S. Pat. No. 5,520,607, may be used.

In some embodiments of the invention, rigid port 148 is formed of a semi-rigid material, such as PVC, which is rigid relative to the material of sheath 125, although being flexible. Alternatively, rigid port 148 is formed of a more rigid material, such as polycarbonate.

At distal end 133, the inner diameter of sheath 125 is optionally substantially equal to the outer diameter of insertion tube 102 with a minimal required addition for channel tube 130. In FIG. 1B, the diameter of sheath 125 is exaggerated for clarity of the figure. Optionally, when a channel tube 130 is not used, the inner diameter of sheath 125 at distal end 133 is substantially equal to the outer diameter of insertion tube 102.

In some embodiments of the invention, in order to allow simpler mounting of sheath 125 over insertion tube 102, a portion 144 of sheath 125 is tapered, such that the distal end 133 of sheath 125 has a smaller diameter than the proximal end 137 of portion 144 of the sheath. Optionally, the tapering proceeds linearly over the tapered portion 144. Alternatively, the tapering proceeds differently in a plurality of sub-segments of portion 144. For example, a first sub-segment close to distal end 133 optionally has a higher degree of tapering than a more proximal sub-segment.

The tapering of portion 144 is optionally less than 0.3 degrees on each side, for example about 0.2 degrees. In some embodiments of the invention, the tapering is of no more than 0.1 degrees or even less than 0.07 degrees. In an exemplary embodiment of the invention, the tapering is of about 0.06 degrees. The use of such a small degree of tapering provides, on the one hand, that there is not too much excess material of sheath 125 inserted into the patient, while enjoying the advantage of easier insertion of tube 102 into sheath 125, provided by the tapering. In other embodiments of the invention, the tapering has a large extent, more than 0.5 degrees or even more than 2 degrees. In an exemplary embodiment of the invention, the sheath has a tapering of above 5 degrees, for example between 6-7 degrees. Such a large tapering allows a very simple insertion of the insertion tube into the sheath, and may be used when the close fitting of the sheath on the insertion tube is not important. Other considerations used in selecting the angle of tapering optionally include, the length of the sheath and/or the stickiness of the surface of the sheath that contacts the insertion tube.

In some embodiments of the invention, portion 144 has a length of between about 320-400 millimeters (e.g., 350 millimeters) and the diameter d2 at proximal end 137 is larger than the diameter d1 at distal end 133 by less than 2 millimeters, or even less than a single millimeter. In other embodiments of the invention, sheath 125 is longer than 50 centimeters, longer than 70 centimeters or even longer than 90 centimeters.

Optionally, sheath 125 is produced using an accurate production method, such as the heat forming method of U.S. Pat. No. 6,733,440, so as to achieve the desired small tapering extent. In some embodiments of the invention, a heated Teflon-coated mandrel with the desired tapering of the sheath 125 is used in forming the sheath. Alternatively, sheath 125 is produced using any other method, such as a plastic extrusion method.

In some embodiments of the invention, sheath 125 is formed of an elastic material which allows stretching of the sheath for insertion of insertion tube 102 into the sheath. Alternatively, sheath 125 is non-elastic, and the tapering makes the insertion of tube 102 easier.

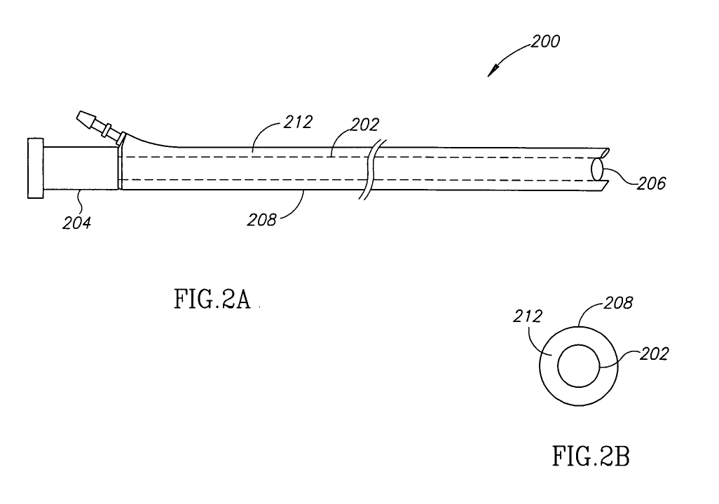

FIGS. 2A and 2B are a schematic side view and a cross sectional view of a sheath assembly 200, in accordance with an exemplary embodiment of the present invention. Assembly 200 optionally includes an internal sheath 202 adapted to receive an endoscope and isolate the endoscope from the environment. In some embodiments of the invention, a relatively rigid pipe section 204 is located at a proximal end of internal sheath 202, to aid insertion of the endoscope into the internal sheath. Pipe section 204 may be of substantially any desired rigidity level, such as discussed above regarding rigid port 148. An optional sealed window 206 at the distal end of internal sheath 202, possibly isolates the endoscope from the environment while allowing a camera or fiberoptic image bundle of the endoscope to provide images of the tissue external to sheath assembly 200.

An external sheath 208, having a larger circumference than internal sheath 202, optionally surrounds internal sheath 202. During insertion of an endoscope covered by sheath assembly 200 into a patient, external sheath 208 is optionally closely folded around internal sheath 202, such that the cross-sectional area of an endoscope with sheath assembly 200 is not substantially enlarged by the inclusion of external sheath 208. After sheath assembly 200 is inserted into the patient, external sheath 208 is unfolded, to form a channel 212 in the area between internal sheath 202 and external sheath 208, such that at least a portion of external sheath 208 serves as a channel tube defining channel 212.

In an exemplary embodiment of the invention, internal sheath 202 has an outer diameter of between about 3.5-3.7 mm and the inner diameter of external sheath 208 is larger than the outer diameter of internal sheath 202 by about 0.25 mm. In other embodiments of the invention, smaller and/or larger diameters of the inner sheath, are used. Optionally, the inner diameter of internal sheath 202 is selected to snugly fit over the endoscope. Alternatively, internal sheath 202 has a larger diameter than the endoscope, such that the volume between internal sheath 202 and the endoscope can be used for a working channel and/or to allow relatively easy sliding of internal sheath 202 over the endoscope.

In some embodiments of the invention, the inner diameter of external sheath 208 is larger than the outer diameter of internal sheath 202 by more than 0.25 mm, for example by between about 0.5-1 mm or even more, depending on the usage of the channel. In an exemplary embodiment of the invention, the diameter of external sheath 208 is greater than the diameter of internal sheath 202 by between about 2-3 mm, for example 2.5 mm, so that a sufficiently large working tube, can be located-in or passed through channel 212. For example, channel 212 may be used to carry a suction tube, such as described in PCT publication WO2005/027999, filed Sep. 20, 2004 and/or in U.S. provisional patent application 60/503,780, titled “Braided Minimally Invasive Channel” and filed Sep. 18, 2003, the disclosures of which are incorporated herein by reference. The braided working channel is optionally longer than internal sheath 202, such that if internal sheath 202 is stretched it is not retracted beyond the proximal end of the braided channel.

In some embodiments of the invention, both internal sheath 202 and external sheath 208 are tapered to a same extent. Alternatively, internal sheath 202 is tapered with a greater angle than external sheath 208, as the tapering of internal sheath 202 is more important for the ease of insertion of an endoscope into sheath assembly 200. In some embodiments of the invention, external sheath 208 is not tapered and only internal sheath 202 is tapered.

Further alternatively or additionally, internal sheath 202 is tapered to a lesser extent than external sheath 208, or is not tapered at all. The internal sheath 202 thus closely contacts the endoscope, while the external sheath 208 allows for leeway in the location of the braided channel, or any other auxiliary tube located between the internal and external sheaths. In some embodiments of the invention, the auxiliary tube is longer than external sheath 208 and/or internal sheath 202, and may form an “S” curve along its length. The extra volume provided by the tapering of external sheath 208, allows room for the curving of the tube without blocking the internal sheath 202 and interfering with the insertion of the endoscope.

Sheaths 202 and 208 have a thickness of between about 0.03 to 0.4 mm, for example between about 0.05 to 0.12 mm. Alternatively, thicker or thinner sheath materials may be used.

Channel 212 is optionally used to provide fluids to the distal end of sheath assembly 200. Alternatively or additionally, channel 212 is used for introducing accessory devices. Further alternatively or additionally, a working tube is introduced to the patient through channel 212. Optionally, the working tube is relatively rigid, so that the channel does not collapse on the working tube.

The size difference between the circumferences of internal sheath 202 and external sheath 208 is optionally chosen according to a desired size of channel 212. In an exemplary embodiment of the invention, the size of the channel 212 is chosen according to a desired impedance for fluids passing through the channel. In some embodiments of the invention, the size of channel 212 is chosen as a compromise between maximizing the fluid impedance and minimizing the cross-sectional area of sheath assembly 200. Alternatively or additionally, the size of channel 212 is set to achieve a predetermined fluid impedance.

Sheath 208 optionally comprises an elastic material, such as polyurethane or polyvinylchloride with a sufficiently large amount of added plasticizer, that allows sheath 208 to bend longitudinally around corners while the sheathed endoscope is inserted into the patient. Alternatively, the material of external sheath 208 is relatively non-elastic, e.g., Polyethyleneterephtalate (PET), polyvinylchloride with a relatively small amount of added plasticizer, or a very thin (e.g., between about 0.05-0.1 mm) layer of Teflon or Polyethylene, as relatively non-elastic materials are generally more suitable for folding and for passing tubes and/or tools through them, due to their relative stiffness. In some embodiments of the invention, the elasticity of the material is chosen as a compromise between the desire for smooth bending and the easier folding and/or passing of tools.

In some embodiments of the invention, sheath 208 is formed in a self-collapsible manner, such that when not held open, channel 212 closes. Alternatively, sheath 208 is formed in a non-self-collapsible manner, such that once opened channel 212 does not close unless a force to induce the collapse is applied to the channel. For example, the material of sheath 208 may be deformed in a predetermined shape, as is known in the art of stents, such that it does not collapse after being unfolded. Optionally, in order to prevent collapse, sheath 208 is deformed over its entire length. Alternatively, sheath 208 is deformed in one or more locations along its length, which locations are sufficient to prevent collapse of channel 212. Further alternatively, stent-like structures are embedded within sheath 208 along its length in order to prevent collapse after it is unfolded.

Internal sheath 202 optionally comprises the same material and/or has the same thickness as sheath 208, possibly allowing a simpler production procedure. Alternatively, internal sheath 202 and external sheath 208 comprise different materials. For example, internal sheath 202 may comprise a thinner or weaker material as it is less affected by the forces involved in inserting the sheath assembly to a patient. Alternatively, internal sheath 202 is relatively rigid and/or stronger than the external sheath, for example reinforced by relatively rigid rings, in order to prevent internal sheath 202 being affected when channel 212 is being used.

In some embodiments of the invention, the entire annular cross-section between internal sheath 202 and external sheath 208 forms channel 212 and is open, for example, for flow of fluids. Using an annular channel is relatively immune against blockage due to bending of sheath 208, as a bend in one direction still allows passage of fluids on the opposite side of the annular channel. Alternatively, internal sheath 202 is fastened to external sheath 208 along one or more longitudinal lines or portions. The fastening of the internal sheath 202 to external sheath 208 optionally limits the size of channel 212. Alternatively or additionally, the fastening of sheaths 202 and 208 to each other does not necessarily limit the size of channel 212, but simplifies the combined insertion of the sheaths and/or prevents distortion of the sheath assembly during insertion. The fastening of the internal sheath 202 to external sheath 208 optionally also prevents a working tube or tool passed through channel 212 from inadvertently wrapping around the internal sheath during insertion to the channel.

Alternatively to including a single channel 212, in some embodiments of the invention, channel 212 is divided along its entire length into a plurality of sub-channels. Further alternatively or additionally, channel 212 is divided into a plurality of sub-channels over only a portion of the length of sheath assembly 200. For example, when the separate sub-channels are used for leading separate working tubes, the sub-channels are optionally defined at the proximal end of the channel, while at the distal end channel 212 is not divided into sub-channels.

A procedure of mounting sheath 125 or sheath assembly 200 on an endoscope, optionally includes providing the sheath in a laid out, non everted, orientation, before beginning insertion of the endoscope into the sheath and/or mounting the sheath on the endoscope. In some embodiments of the invention, the sheath or sheath assembly is provided in a sterilized package in the laid out orientation. The sheath is optionally held from a relatively rigid proximal port and the endoscope is inserted into the sheath and/or the sheath is pulled over the endoscope. Alternatively or additionally, the mounting may be performed using a holding assembly.

While in a simple embodiment the sheath tapers at a same degree along the entire length of the tapered portion, in other embodiments the angle of the taper is different in different portions along the length of the sheath. For example, the distal end of the sheath optionally has a lower tapering angle than a proximal portion of the sheath, so that the distal end of the insertion tube tightly fits in the sheath.

For simplicity, the diameter was used to state the size of the cross section of the sheath. It is noted, however, that the present invention is not limited to sheaths with a circular cross-section area and the sizes stated in diameters should be interpreted for non-circular embodiments as relating to equivalent areas.

The above description relates to an invasive medical probe which is not tapered and the tapering of the sheath increases the difference in the diameter between the invasive probe and the sheath, along the axis of the invasive probe. It is noted, however, that the principals of the invention may be applied also to tapered elongate medical probes, in which case, the tapering of the sheath is relative to the outer surface of the medical probe. Furthermore, the tapering optionally affects the inner and outer walls of the sheath to a same extent. Alternatively, the inner and outer walls taper to different extents, such that the thickness of the sheath varies along its axial length.

It will be appreciated that the above-described methods may be varied in many ways, for example by using a stepped sheath rather than a tapered sheath. It should also be appreciated that the above described description of methods and apparatus are to be interpreted as including apparatus for carrying out the methods, and methods of using the apparatus.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art. Furthermore, the terms “comprise,” “include,” “have” and their conjugates, shall mean, when used in the claims, “including but not necessarily limited to.” It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

1. A sheath apparatus for an invasive probe, comprising:
a protective sheath, adapted to receive an insertion tube of an endoscope,
wherein the protective sheath is tapered over most of its length with a tapering of less than 0.2 degrees, but more than 0.001 degrees.

2. A sheath apparatus according to claim 1, wherein the protective sheath is tapered over most of its length with a tapering not greater than 0.1 degrees.

3. A sheath apparatus according to claim 1, wherein the protective sheath is tapered over most of its length with a tapering not greater than 0.06 degrees.

4. A sheath apparatus according to claim 1, wherein the protective sheath is thinner than 0.2 mm.

5. A sheath apparatus according to claim 1, comprising an additional external sheath, which surrounds the protective sheath.

6. A sheath apparatus according to claim 5, wherein the external sheath is tapered over most of its length with a tapering extent substantially the same as the protective sheath.

7. A sheath apparatus according to claim 5, wherein the external sheath is tapered over most of its length with a tapering extent smaller than that of the protective sheath.

8. A sheath apparatus according to claim 5, comprising at least one channel tube between the protective sheath and the external sheath.

9. A sheath apparatus according to claim 1, wherein the protective sheath has a length of at least 300 millimeters.

10. A method of inserting an endoscope into a protective sheath, comprising:
providing an insertion tube of an endoscope; and
covering the insertion tube with a protective sheath, which is tapered over most of its length, without everting the sheath in the process of covering the insertion tube.

11. A method according to claim 10, wherein covering the insertion tube comprises covering with at least two sheaths, one of which is within the other.

12. A method according to claim 10, wherein all of the at least two sheaths are tapered at a same extent.

13. A method according to claim 10, wherein covering the insertion tube with a protective sheath, comprises inserting the endoscope into the protective sheath.

14. A method according to claim 11, wherein covering the insertion tube with a protective sheath, comprises pulling the protective sheath over the insertion tube.

15. A sheath apparatus for an invasive probe, comprising:
a tapered protective sheath, adapted to receive an insertion tube of an endoscope; and
a proximal port, which is rigid relative to the sheath, attached to a proximal end of the protective sheath.

16. A sheath apparatus according to claim 15, wherein the sheath has a length of at least 250 millimeters.

17. A sheath package, comprising:
a sterile package; and
a tapered protective sheath, adapted to receive an insertion tube of an endoscope, which is laid out over substantially its entire length without eversion, within the sterile package.

18. A sheath package according to claim 17, comprising a proximal port, which is rigid relative to the sheath, attached to a proximal end of the protective sheath.
